(19) United States
(12) Patent Application Publication
(10) Pub. No.: US 2023/0111691 A1
(43) Pub. Date: Apr. 13, 2023

(54) COMPUTER-READABLE RECORDING MEDIUM STORING COMPOUND SUBSTITUTION PROGRAM, METHOD, AND DEVICE

(71) Applicant: FUJITSU LIMITED, Kawasaki-shi (JP)

(72) Inventor: Kazunari TANAKA, Kawasaki (JP)

(73) Assignee: FUJITSU LIMITED, Kawasaki-shi (JP)

(21) Appl. No.: 18/065,271

(22) Filed: Dec. 13, 2022

Related U.S. Application Data
(63) Continuation of application No. PCT/JP2020/028718, filed on Jul. 27, 2020.

(51) Int. Cl. G16C 20/40; G16C 20/80

(52) U.S. Cl. CPC G16C 20/40; G16C 20/80

(57) ABSTRACT

A non-transitory computer-readable recording medium stores a compound substitution program for causing a computer to execute processing including: specifying a first partial structure included in a first compound; referring to information that indicates a relationship between a plurality of partial structures and selecting a second partial structure related to the first partial structure; specifying a bonding position in the second partial structure, based on a rational formula of the selected second partial structure; and generating information that indicates a second compound obtained by substituting the first partial structure of the first compound with the second partial structure, based on the specified bonding position.

### CROSS-REFERENCE TO RELATED APPLICATION

This application is a continuation application of International Application PCT/JP2020/028718 filed on Jul. 27, 2020 and designated the U.S., the entire contents of which are incorporated herein by reference.

### FIELD

The embodiment discussed herein is related to a compound substitution technology.

### BACKGROUND

In the field of chemistry, there is a case where documents such as patent publications or papers are searched by specifying a compound name as a key. At this time, it is useful to obtain documents regarding not only a compound indicated by the compound name specified as a key and but also compounds having similar structures with the compound. For this, traditionally, a technique has been proposed for specifying a compound that has a similar structure to the compound indicated by the compound name specified as a key and searching for a document regarding the specified compound.

International Publication Pamphlet No. WO 2018/158916, Japanese Laid-open Patent Publication No. 2007-277188, and Japanese Laid-open Patent Publication No. 2019-74843 are disclosed as related art.

### SUMMARY

According to an aspect of the embodiments, a non-transitory computer-readable recording medium stores a compound substitution program for causing a computer to execute processing including: specifying a first partial structure included in a first compound; referring to information that indicates a relationship between a plurality of partial structures and selecting a second partial structure related to the first partial structure; specifying a bonding position in the second partial structure, based on a rational formula of the selected second partial structure; and generating information that indicates a second compound obtained by substituting the first partial structure of the first compound with the second partial structure, based on the specified bonding position.

The object and advantages of the invention will be realized and attained by means of the elements and combinations particularly pointed out in the claims.

It is to be understood that both the foregoing general description and the following detailed description are exemplary and explanatory and are not restrictive of the invention.

### BRIEF DESCRIPTION OF DRAWINGS

FIG. 1 is a diagram illustrating a configuration example of a compound substitution device;

FIG. 2 is a diagram for explaining processing for obtaining compounds having similar structures;

FIG. 3 is a diagram illustrating an example of a steric structure of a compound;

FIG. 4 is a diagram illustrating an example of the steric structure of the compound;

FIG. 5 is a diagram for explaining modeling of a steric structure;

FIG. 6 is a diagram for explaining mapping of a steric structure onto a plane;

FIGS. 7A and 7B are a flowchart illustrating a flow of processing for obtaining similar compounds; and

FIG. 8 is a diagram for explaining a hardware configuration example.

### DESCRIPTION OF EMBODIMENTS

However, the traditional technology has a problem in that it may be difficult to prevent substitution to a non-existent compound.

For example, according to the traditional technology, by substituting a partial structure of a first compound with a partial structure corresponding to a subordinate concept belonging to the same superordinate concept, it is possible to obtain a second compound having a structure similar to the first compound. For example, a similar compound can be obtained by substituting propyl of “2,2-bis(4-hydroxyphenyl)propane” (also known as: bisphenol A) with another alkyl group.

Here, according to the traditional technology, a compound called “2,2-bis(4-hydroxyphenyl)butane” in which a propyl of the bisphenol A is simply substituted with a butyl is obtained. On the other hand, with the traditional technology, there is a case where it is not guaranteed that a compound named “2,2-bis(4-hydroxyphenyl)butane” according to naming rules can actually exist.

In one aspect, an object is to prevent substitution to a non-existent compound.

Hereinafter, an embodiment of a compound substitution program, method, and device will be described in detail with reference to the drawings. Note that the embodiment does not limit the present invention. Furthermore, the individual embodiments may be appropriately combined within a range without inconsistency.

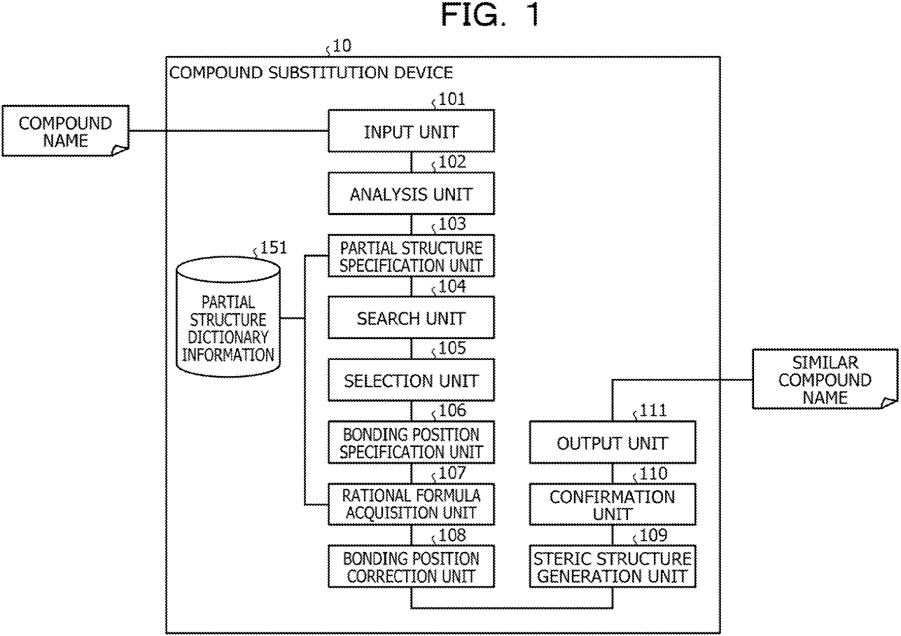

A configuration of a compound substitution device according to the embodiment will be described with reference to FIG. 1. FIG. 1 is a diagram illustrating a configuration example of the compound substitution device. As illustrated in FIG. 1, a compound name is input to a compound substitution device 10. Furthermore, the compound substitution device 10 outputs a similar compound name.

As illustrated in FIG. 1, the compound substitution device 10 includes an input unit 101, an analysis unit 102, a partial structure specification unit 103, a search unit 104, a selection unit 105, a bonding position specification unit 106, a rational formula acquisition unit 107, and a bonding position correction unit 108. Furthermore, the compound substitution device 10 includes a steric structure generation unit 109, a confirmation unit 110, and an output unit 111. Furthermore, the compound substitution device 10 stores partial structure dictionary information 151.

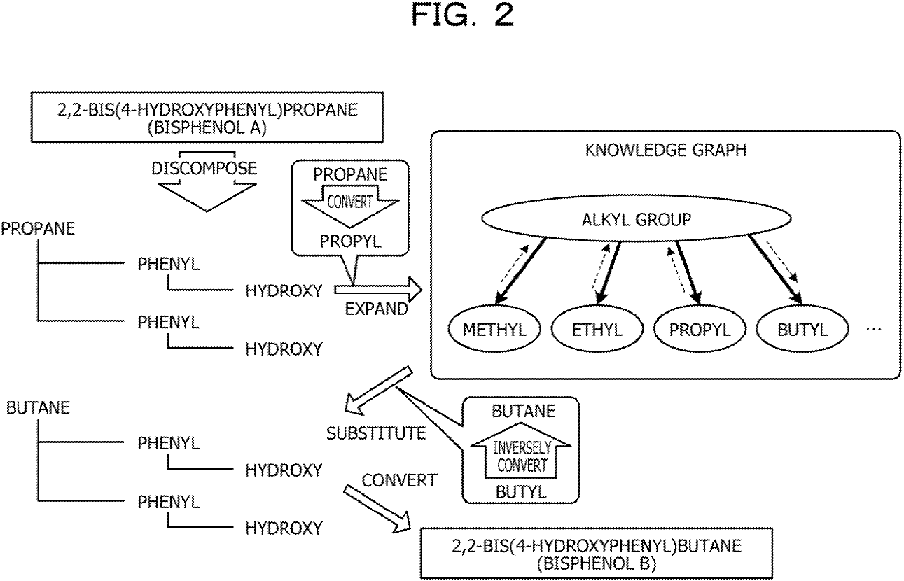

The input unit 101 receives an input of a compound name. The analysis unit 102 analyzes the input compound name. For example, as illustrated in FIG. 2, the analysis unit 102 develops a compound indicated by the input compound name into a partial structure. FIG. 2 is a diagram for explaining processing for obtaining compounds having similar structures.

In the example in FIG. 2, the input unit 101 receives an input of a character string of “2,2-bis(4-hydroxyphenyl)propane”. 2,2-bis(4-hydroxyphenyl)propane is an example of a first compound.

The analysis unit 102 obtains a structure in which two phenyls are bonded to propane and hydroxy is further bonded to each phenyl, based on the character string “2,2-bis(4-hydroxyphenyl)propane”. As illustrated in FIG. 2, the analysis unit 102 may represent the structure with tree-format data.

The partial structure specification unit 103 specifies a first partial structure included in the first compound. For example, the partial structure specification unit 103 can specify a partial structure that has an effect on properties, as small as possible, as a compound when the partial structure is substituted with another partial structure, as the first partial structure. In the example in FIG. 2, the partial structure specification unit 103 specifies propane as the first partial structure.

The search unit 104 searches a knowledge graph using the first partial structure as a key. The knowledge graph is graph representing a relationship between a superordinate concept and a subordinate concept of a partial structure of a compound. The knowledge graph in FIG. 2 indicates that methyl, ethyl, propyl, and butyl exist as subordinate concepts of an alkyl group. For example, the knowledge graph in FIG. 2 indicates that an alkyl group is a common superordinate concept of methyl, ethyl, propyl, and butyl.

The search unit 104 searches a knowledge graph by converting a name of the first partial structure of the compound name into a name of a substituent. In the example in FIG. 2, the search unit 104 converts “propane” that is the name of the first partial structure into “propyl” that is a name of a corresponding substituent.

The selection unit 105 refers to information indicating a relationship between a plurality of partial structures and selects a second partial structure related to the first partial structure. The information indicating the relationship between the plurality of partial structures is, for example, a set of subordinate concepts having the alkyl group in the knowledge graph as the superordinate concept. For example, the selection unit 105 selects butyl as a second partial structure related to propyl.

Moreover, the selection unit 105 inversely converts “butyl” that is the name of the selected second partial structure into “butane” that is a name of the partial structure of the compound. As a result, the selection unit 105 obtains a structure in which two phenyls are bonded to butane and hydroxy is further bonded to each phenyl.

When “propane” in the name of the first compound is simply substituted with “butane”, a name of a compound indicated by the structure obtained by the selection unit 105 can be written as “2,2-bis(4-hydroxyphenyl)butane” (also known as: bisphenol B). A compound called 2,2-bis(4-hydroxyphenyl)butane exists.

Here, “2,2-bis(4-hydroxyphenyl) X” means that both of bonding positions of two 4-hydroxyphenyls to an alkyl group X are the second carbon. Based on this, a case will be considered where the selection unit 105 selects methane, not butane, as the second partial structure. At this time, by executing similar processing as in a case where butane is selected, a name of a compound “2,2-bis(4-hydroxyphenyl)methane” is obtained.

On the other hand, because methane includes only one carbon, there is a contradiction in the name “2,2-bis(4-hydroxyphenyl)methane”. Therefore, the compound having the name of “2,2-bis(4-hydroxyphenyl)methane” cannot exist. Therefore, the compound substitution device 10 obtains an existable compound that has a structure in which two phenyls are bonded to methane and hydroxy is further bonded to each phenyl through processing to be described below.

The bonding position specification unit 106 specifies a bonding position in the second partial structure, based on a rational formula of the selected second partial structure. The rational formula is acquired from the partial structure dictionary information 151 by the rational formula acquisition unit 107. The partial structure dictionary information 151 is information in which a name of a partial structure is associated with a rational formula.

For example, a rational formula of methane is CH4, and up to four hydrogens can be extracted from the first carbon. Therefore, the bonding position specification unit 106 specifies that a bonding position of methane is the first carbon.

Furthermore, for example, a rational formula of ethane is CH3CH3, and up to three hydrogens can be extracted from each carbon. Therefore, the bonding position specification unit 106 specifies that bonding positions of ethane are the first and second carbons. Furthermore, for example, a rational formula of butane is CH3CH2CH2CH3, and at least two hydrogens can be extracted from each carbon. Therefore, the bonding position specification unit 106 specifies that bonding positions of butane are the first to four carbons. In this way, the bonding position specification unit 106 can specify candidates of the plurality of bonding positions, based on types and valences of atoms included in the selected second partial structure.

As described above, the compound having the name of “2,2-bis(4-hydroxyphenyl)methane” cannot exist. Therefore, the bonding position correction unit 108 corrects the name of the compound to a name of an existable compound “1,1-bis(4-hydroxyphenyl)methane”, based on the bonding position specified by the bonding position specification unit 106. Furthermore, because methane includes only one carbon, “1,1-” in “1,1-bis(4-hydroxyphenyl)methane” may be omitted. The name of the compound in that case is “bis(4-hydroxyphenyl)methane”.

The steric structure generation unit 109 generates information indicating a second compound obtained by substituting the first partial structure of the first compound with the second partial structure, based on the specified bonding position. Furthermore, the confirmation unit 110 confirms whether or not the second compound obtained by substituting the first partial structure of the first compound with the second partial structure based on the specified bonding position can exist as a steric structure. Moreover, in a case where it is confirmed that the second compound can exist as the steric structure, the steric structure generation unit 109 generates information indicating the second compound.

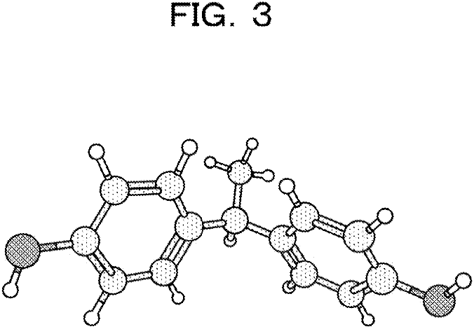

In a case where the second partial structure is ethane, the second compound includes 1,1-bis(4-hydroxyphenyl)ethane (also known as: bisphenol E), 1,2-bis(4-hydroxyphenyl)ethane, and 2,2-bis(4-hydroxyphenyl)ethane. As an example, a steric structure of 1,1-bis(4-hydroxyphenyl)ethane is illustrated in FIG. 3. FIG. 3 is a diagram illustrating an example of a steric structure of a compound.

The confirmation unit 110 confirms whether or not partial structures collide with each other or the like, in consideration of positions and sizes of atoms, based on the steric structure as illustrated in FIG. 3. Accordingly, the confirmation unit 110 checks a possibility of existence of the compound.

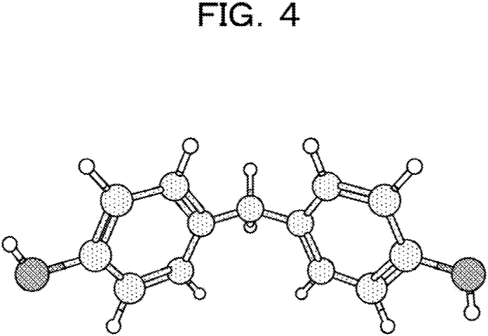

A steric structure of “1,1-bis(4-hydroxyphenyl)methane” that is the name of the compound obtained by the bonding position correction unit 108 is illustrated in FIG. 4. FIG. 4 is a diagram illustrating an example of a steric structure of a compound.

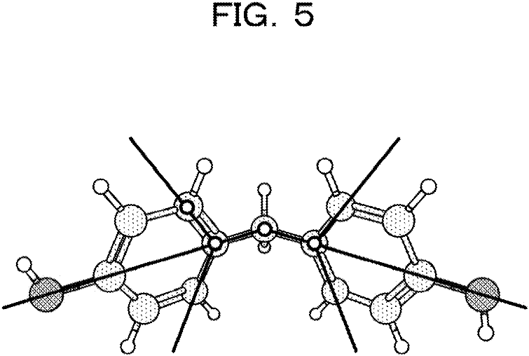

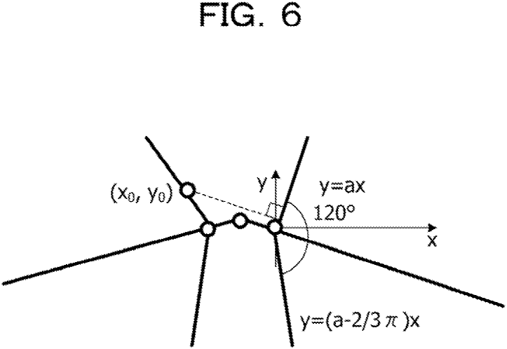

Here, as illustrated in FIG. 5, the confirmation unit 110 samples a carbon skeleton and models carbons as points and bonds as lines. FIG. 5 is a diagram for explaining modeling of a steric structure. Moreover, as illustrated in FIG. 6, the confirmation unit 110 maps the steric structure onto a plane so as to maximize an angle between lines. FIG. 6 is a diagram for explaining mapping of a steric structure onto a plane. Note that, since the benzene ring is hexagonal, an angle between carbon atoms on the plane is 120°. In this way, the confirmation unit 110 can map an originally three-dimensional object to a two-dimensional object by changing a scope depending on a sampling method.

Moreover, as illustrated in FIG. 6, the confirmation unit 110 sets a coordinate system of the mapped plane, and calculates a distance d between a point (x₀, y₀) and a line y = ax as in the formula (1).

$$d = \frac{\left|a {x}_{0} - {y}_{0}\right|}{\sqrt{{a}^{2} + 1}}$$

When there is a of which a distance is equal to or more than a carbon radius exists for all the points, the confirmation unit 110 determines that there is no collision between atoms in the second compound. The output unit 111 outputs the name of the second compound that is determined, by the confirmation unit 110, to have no collision between the atoms as the similar compound name of the first compound. For example, the compound substitution device 10 can receive an input of the character string of “2,2-bis(4-hydroxyphenyl)propane” and output the character string of “1,1-bis(4-hydroxyphenyl)ethane”.

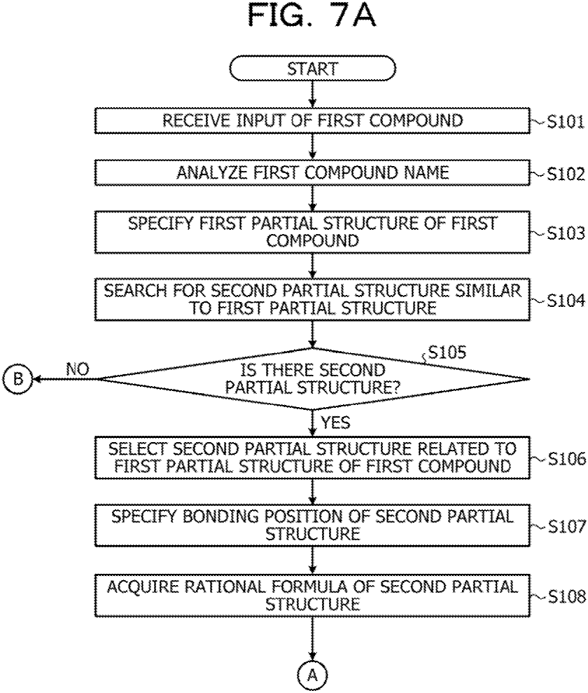

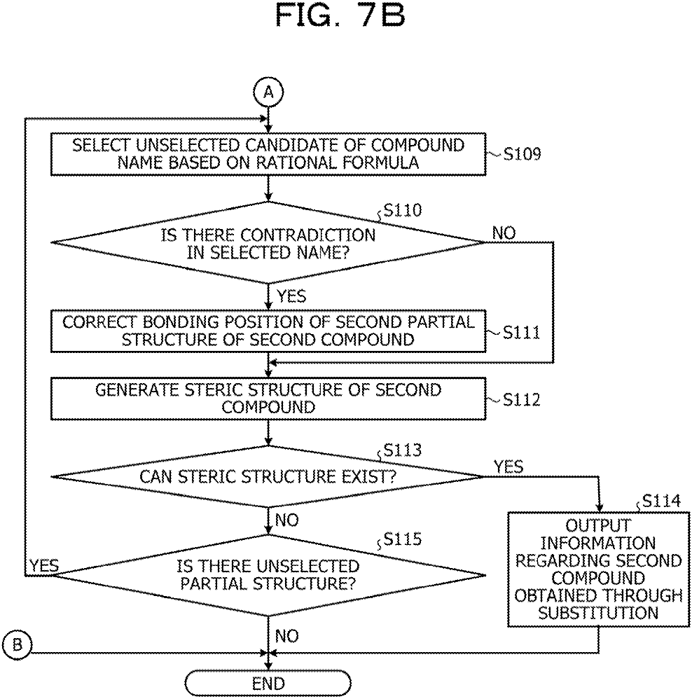

FIGS. 7A and 7B are a flowchart illustrating a flow of processing for obtaining similar compounds. As illustrated in FIGS. 7A and 7B, first, the input unit 101 receives an input of the first compound (step S101). Next, the analysis unit 102 analyzes the first compound name (step S102). Then, the partial structure specification unit 103 specifies the first partial structure of the first compound (step S103).

The search unit 104 searches for the second partial structure similar to the first partial structure (step S104). In a case where there is no second partial structure as a result of the search (step S105, No), the compound substitution device 10 ends the processing. On the other hand, in a case where there is the second partial structure as a result of the search (step S105, Yes), the selection unit 105 selects the second partial structure related to the first partial structure of the first compound (step S106).

Here, the bonding position specification unit 106 specifies the bonding position of the second partial structure (step S107). Then, the rational formula acquisition unit 107 acquires the rational formula of the second partial structure (step S108).

The bonding position correction unit 108 selects an unselected candidate of a compound name based on the rational formula (step S109). In a case where there is a contradiction in the selected name (step S110, Yes), the bonding position correction unit 108 corrects the bonding position of the second partial structure of the second compound (step S111). On the other hand, in a case where there is no contradiction in the selected name (step S110, No), the processing proceeds to the steric structure generation unit 109.

The steric structure generation unit 109 generates the steric structure of the second compound (step S112). Here, the confirmation unit 110 confirms whether or not the steric structure can exist (step S113). In a case where the steric structure cannot exist (step S113, No), the confirmation unit 110 proceeds to step S115. On the other hand, in a case where the steric structure can exist (step S113, Yes), the output unit 111 outputs information regarding the second compound obtained through substitution (step S114).

In a case where there is an unselected partial structure (step S115, Yes), the bonding position correction unit 108 returns to step S109 and repeats the processing. In a case where there no unselected partial structure (step S115, No), the compound substitution device 10 ends the processing.

As described above, the partial structure specification unit 103 specifies the first partial structure included in the first compound. The selection unit 105 refers to information indicating a relationship between a plurality of partial structures and selects a second partial structure related to the first partial structure. The bonding position specification unit 106 specifies a bonding position in the second partial structure, based on a rational formula of the selected second partial structure. The steric structure generation unit 109 generates information indicating a second compound obtained by substituting the first partial structure of the first compound with the second partial structure, based on the specified bonding position. In this way, the compound substitution device 10 can generate the information considering the steric structure of the compound whose partial structure has been substituted. As a result, according to the present embodiment, substitution to a non-existent compound can be prevented.

The bonding position specification unit 106 specifies the candidates of the plurality of bonding positions, based on the types and the valences of the atoms included in the selected second partial structure. As a result, it is possible to exclude a compound having a contradictory structure and obtain a compound that can exist as a candidate.

The confirmation unit 110 confirms whether or not the second compound obtained by substituting the first partial structure of the first compound with the second partial structure based on the specified bonding position can exist as a steric structure. In a case where it is confirmed that the second compound can exist as the steric structure, the steric structure generation unit 109 generates the information indicating the second compound. In this way, the confirmation unit 110 confirms whether or not the second compound can exist as the steric structure. As a result, a compound that cannot exist can be excluded.

The present embodiment is effective, for example, in a case where search for a document is performed using a compound name. In document search in the field of chemistry, there is a case where it is desired to consider a different notation (another name, chemical formula, SMILES, or the like) of a compound of which a name is input as a keyword and a compound that has a similar structure or property that does not completely match the structure.

For example, if a compound similar to the input compound can be searched as including the similar compound as a key, this is effective in a case where a similarity between patent documents is determined. On the other hand, for example, in patent documents in the field of chemistry, there is a case where a large number of compounds are used in association with each other with a list of compound names, Markush claims, or the like, and it is considered to obtain a more useful search result by capturing these as a compound group at the time of the search. Furthermore, there is a case where an entire compound group is written in the Markush format in patent documents and only the small number of individual specific compound names are written. Moreover, in a case where search is performed using the compound name, to define a compound group including the compound name needs specialized knowledge, time, and labor. When any oversight occurs, this causes search omissions.

According to the present embodiment, for example, a name of a similar compound “1,1-bis(4-hydroxyphenyl)methane” can be obtained with respect to the input of “2,2-bis(4-hydroxyphenyl)propane”. At this time, a compound that cannot exist is excluded from the similar compounds. As a result, according to the present embodiment, a name of a compound that can be used as a keyword to obtain a more useful search result can be obtained.

Pieces of information including a processing procedure, a control procedure, a specific name, various types of data, and parameters described above or illustrated in the drawings may be optionally changed unless otherwise specified. Furthermore, the specific examples, distributions, numerical values, and the like described in the embodiment are merely examples, and may be changed in any ways.

Furthermore, each component of each device illustrated in the drawings is functionally conceptual, and is not necessarily physically configured as illustrated in the drawings. For example, specific forms of distribution and integration of each device are not limited to those illustrated in the drawings. For example, all or a part of the devices may be configured by being functionally or physically distributed or integrated in any units according to various types of loads, usage situations, or the like. Moreover, all or any part of individual processing functions performed in each device may be implemented by a central processing unit (CPU) and a program analyzed and executed by the CPU, or may be implemented as hardware by wired logic.

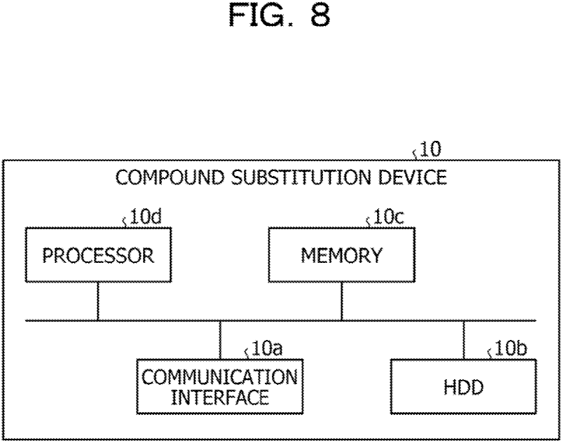

FIG. 8 is a diagram for explaining a hardware configuration example. As illustrated in FIG. 8, the compound substitution device 10 includes a communication interface 10a, a hard disk drive (HDD) 10b, a memory 10c, and a processor 10d. Furthermore, the individual units illustrated in FIG. 8 are mutually connected to each other by a bus or the like.

The communication interface 10a is a network interface card or the like and communicates with another server. The HDD 10b stores a program that activates the functions illustrated in FIG. 1, and a DB.

The processor 10d is a hardware circuit that reads, from the HDD 10b or the like, a program for executing the processing of each processing unit illustrated in FIG. 1, and loads it into the memory 10c to operate a process that executes each function described with reference to FIG. 1 or the like. For example, with this process, a function of each processing unit included in the compound substitution device 10 is implemented. For example, the processor 10d reads programs for implementing the functions of the analysis unit 102, the search unit 104, the selection unit 105, the bonding position specification unit 106, the bonding position correction unit 108, the steric structure generation unit 109, the confirmation unit 110, or the like from the HDD 10b or the like. Then, the processor 10d implements the analysis unit 102, the search unit 104, the selection unit 105, the bonding position specification unit 106, the bonding position correction unit 108, the steric structure generation unit 109, the confirmation unit 110, or the like based on a plurality of instructions included in the program.

In this way, the compound substitution device 10 operates as an information processing device that performs a compound substitution method by reading and executing the program. Furthermore, the compound substitution device 10 may implement functions similar to those of the embodiment described above by reading the program described above from a recording medium with a medium reading device and executing the read program described above. Note that other programs referred to in the embodiments are not limited to being executed by the compound substitution device 10. For example, the embodiment may be similarly applied to a case where another computer or server executes the program, or to a case where such computer and server cooperatively execute the program.

This program may be distributed via a network such as the Internet. Furthermore, this program may be recorded on a computer-readable recording medium such as a hard disk, flexible disk (FD), compact disc read only memory (CD-ROM), magneto-optical disk (MO), or digital versatile disc (DVD) and may be executed by being read from the recording medium by a computer.

All examples and conditional language provided herein are intended for the pedagogical purposes of aiding the reader in understanding the invention and the concepts contributed by the inventor to further the art, and are not to be construed as limitations to such specifically recited examples and conditions, nor does the organization of such examples in the specification relate to a showing of the superiority and inferiority of the invention. Although one or more embodiments of the present invention have been described in detail, it should be understood that the various changes, substitutions, and alterations could be made hereto without departing from the spirit and scope of the invention.

What is claimed is:

1. A non-transitory computer-readable recording medium storing a compound substitution program for causing a computer to execute processing comprising:
specifying a first partial structure included in a first compound;
referring to information that indicates a relationship between a plurality of partial structures and selecting a second partial structure related to the first partial structure;
specifying a bonding position in the second partial structure, based on a rational formula of the selected second partial structure; and
generating information that indicates a second compound obtained by substituting the first partial structure of the first compound with the second partial structure, based on the specified bonding position.

2. The non-transitory computer-readable recording medium according to claim 1, wherein
the specifying processing includes processing of specifying candidates of a plurality of bonding positions, based on types and valences of atoms included in the selected second partial structure.

3. The non-transitory computer-readable recording medium according to claim 1, for causing the computer to execute processing comprising:
confirming whether or not a second compound obtained by substituting the first partial structure of the first compound with the second partial structure based on the specified bonding positions is existable as a steric structure, wherein
the generating processing is executed in a case where that the second compound is existable as the steric structure is confirmed.

4. A compound substitution method comprising:
specifying a first partial structure included in a first compound;
referring to information that indicates a relationship between a plurality of partial structures and selecting a second partial structure related to the first partial structure;
specifying a bonding position in the second partial structure, based on a rational formula of the selected second partial structure; and
generating information that indicates a second compound obtained by substituting the first partial structure of the first compound with the second partial structure, based on the specified bonding position.

5. A device comprising:
a memory; and
a processor coupled to the memory and configured to:
specify a first partial structure included in a first compound;
refer to information that indicates a relationship between a plurality of partial structures and selecting a second partial structure related to the first partial structure;
specify a bonding position in the second partial structure, based on a rational formula of the selected second partial structure; and
generate information that indicates a second compound obtained by substituting the first partial structure of the first compound with the second partial structure, based on the specified bonding position.
